(19) United States
(12) Patent Application Publication
(10) Pub. No.: US 2022/0230007 A1
(43) Pub. Date: Jul. 21, 2022

(54) Systems and Methods for Labeling, Identifying, and Tracking Data Related to Consumable Product

(71) Applicant: PLAS-TECH ENGINEERING, INC., Lake Geneva, WI (US)

(72) Inventors: AARON HIRSCHMANN, LAKE GENEVA, WI (US); ROBERT FESUS, LAKE GENEVA, WI (US)

(21) Appl. No.: 17/715,745

(22) Filed: Apr. 7, 2022

Related U.S. Application Data
(63) Continuation of application No. 16/176,029, filed on Oct. 31, 2018, now abandoned.
Application No. 16/176,029 is a continuation-in-part of application No. 15/946,173, filed on Apr. 5, 2018, now abandoned.
Application No. 15/946,173 is a continuation of application No. 15/407,379, filed on Jan. 17, 2017, now abandoned.
(60) Provisional application No. 62/278,758, filed on Jan. 14, 2016.

(51) Int. Cl. G06K 7/14; G16H 20/17; G06K 19/06; A01K 5/00; A61D 7/00; G16Z 99/00

(52) U.S. Cl. CPC G06K 7/1417; G06K 7/1413; G16H 20/17; G16Z 99/00; G06K 19/06028; A01K 5/00; A61D 7/00; G06K 19/06037

(57) ABSTRACT

Systems and methods for labeling, identifying, and tracking data related to consumable substance include encoded information related to the substance itself, its container, and/or an animal (e.g., human, feline, canine, livestock) intended to receive the substance in vivo.

### RELATED APPLICATIONS

This application is a continuation of co-pending U.S. Non-Provisional patent application Ser. No. 16/176,029, filed Oct. 31, 2018, and titled, “Systems and Methods for Labeling, Identifying, and Tracking Data Related to Consumable Product,” which is a continuation-in-part of abandoned U.S. Non-Provisional patent application Ser. No. 15/946,173, filed 5 Apr. 2018, and titled, “Systems and Methods for Labeling, Identifying, and Tracking Data Related to Consumable Product,” which is a continuation of abandoned U.S. Non-Provisional patent application Ser. No. 15/407,379, filed 17 Jan. 2017, and titled, “Systems and Methods for Labeling, Identifying, and Tracking Data Related to Consumable Product,” which claims the benefit of now expired U.S. Provisional Patent Application Ser. No. 62/278,758, filed 14 Jan. 2016, and titled “Systems and Methods for Labeling, Identifying, and Tracking Data Related to Consumable Product,” both of which are incorporated herein by reference in their entireties.

### BACKGROUND

This invention relates generally to the labeling, identification, and/or tracking of data related to consumable products, and more particularly to coding and related systems used in marking and tracking in vivo consumable products (e.g., food, medication, vitamins, etc.) and/or their containers.

Assurance as to substances to be consumed within or delivered into an animal body (e.g. a human body) cannot be overstressed. Humans assess items or materials to be consumed or otherwise taken in vivo by many indicators, including visible physical characteristics of a substance, tactile perception, through olfactory analysis, and through taste, usually in that order and sometimes in combination. However, there are times when the identity of a product may not be discernible by any of the senses, or it may be dangerous to do so. Such is the case with pharmaceuticals and fluids to be introduced into an animal body (e.g., introduced orally, percutaneously, and/or intravenously into a human body).

In conventional syringe-filling operations for dosed or pre-filled syringes, for example, a filling machine was generally manually programmed with information such as syringe size (or capacity) and/or dosage volume (e.g., in milliliters). At some time other than the contemporaneous filling process, syringes may be tagged with a label, such as an adhesive label, in an attempt to identify the contents of the syringe. However, because labels were usually affixed to syringes after a manufacturing/filling process, chances of mislabeling and, therefore, substance misidentification increased.

The art of information management, creation, and display in conjunction with food and/or drug administration would be enhanced by systems and methods for logging, verification, and confirmation substantially contemporaneously with administration.

### SUMMARY OF THE INVENTION

The present invention relates to improved systems and methods for preferably permanently (i.e., indelibly) labeling, identifying, and/or tracking information or data related to a consumable, preferably digestible product (e.g., food, medication, vitamins, etc., in liquid or solid form).

An embodiment of a system according to the present invention includes a container for containing a consumable substance and configured to be consumed by an animal to deliver the consumable substance to the animal. The container itself is digestible by the animal. A machine-readable code indelibly placed on the container by laser etching. The machine-readable code is encoded with data representative of information related to at least one of the container, the consumable substance contained within the container, or the animal to which the substance is to be administered.

According to an embodiment of a system according to the present invention, the information related to the consumable substance contained within the container includes at least one of substance ingredients, possible side effects caused by the substance, a substance production date, a substance production time of day, or a substance expiration date.

According to another embodiment of a system according to the present invention, the information related to the container includes at least one of a container capacity, a container type, a manufacturer lot number for the container, a container serial number, a container production date, a container production time of day, a container expiration date, or a list of compatible contents.

According to a further embodiment of a system according to the present invention, the information related to the animal to which the substance is to be administered includes at least one of a postal address, a telephone number, an e-mail address, a health insurance company name, a health insurance policy number, a social security number, a payment account, a birthdate, age, prescribed dosage, or prescribed dosage schedule.

According to yet another embodiment of a system according to the present invention, the machine-readable code may be a one-dimensional machine-readable code (e.g., a conventional bar code) or a two-dimensional machine-readable code (e.g., a QR code).

According to another embodiment of a system according to the present invention, the machine-readable code may be further encoded with data representative of information related to a name of at least one of a prescribing physician, a pharmacist filling a prescription of the consumable substance, or an administering physician.

According to another embodiment of a system according to the present invention, the container comprises a capsule.

According to another embodiment of a system according to the present invention, the capsule comprises a first half and a second half.

According to another embodiment of a system according to the present invention, the machine-readable code is provided on one of the first half or the second half.

According to another embodiment of a system according to the present invention, the machine-readable code is provided on both the first half and the second half.

Another embodiment of a system according to the present invention includes a consumable composition of a substance configured to be consumed by an animal. A machine-readable code indelibly placed on the consumable composition by laser etching. The machine-readable code is encoded with data representative of information related to at least one of the consumable composition or the animal by which the consumable composition is to be consumed.

According to another embodiment of a system according to the present invention, the information related to the consumable composition includes at least one of composition ingredients, possible side effects caused by the composition, a composition production date, a composition production time of day, or a composition expiration date.

According to still another embodiment of a system according to the present invention, the information related to the animal to which the composition is to be administered includes at least one of a postal address, a telephone number, an e-mail address, a health insurance company name, a health insurance policy number, a social security number, a payment account, a birthdate, age, prescribed dosage, or prescribed dosage schedule.

According to yet another embodiment of a system according to the present invention, the machine-readable code may be a one-dimensional machine-readable code (e.g., a conventional bar code) or a two-dimensional machine-readable code (e.g., a QR code).

According to another embodiment of a system according to the present invention, the machine-readable code may be further encoded with data representative of information related to a name of at least one of a prescribing physician, a pharmacist filling a prescription of the consumable substance, or an administering physician.

According to another embodiment of a system according to the present invention, the consumable composition is in the form of a tablet.

### BRIEF DESCRIPTION OF THE DRAWINGS

FIG. 1A is a perspective view of a first embodiment of a container according to the present invention.

FIG. 1B is a zoomed in view of a portion of FIG. 1A.

FIG. 2 is a perspective view of a second embodiment of a container according to the present invention.

FIG. 3 is a perspective view of a third embodiment of a container according to the present invention.

FIG. 4 is a perspective view of a fourth embodiment of a container according to the present invention.

FIG. 5 is a perspective view of a first embodiment of an in vivo consumable container according to the present invention.

FIG. 6 is a perspective view of a first embodiment of an in vivo consumable substance according to the present invention.

FIG. 7 is a perspective view of an embodiment of an administration or consumption environment according to the present invention.

FIG. 8 is a perspective view of a fourth embodiment of a container according to the present invention.

### DETAILED DESCRIPTION

Although the disclosure hereof enables those skilled in the art to practice the invention, the embodiments described merely exemplify the invention which may be embodied in other ways. While the preferred embodiment has been described, the details may be changed without departing from the invention, which is defined by the claims.

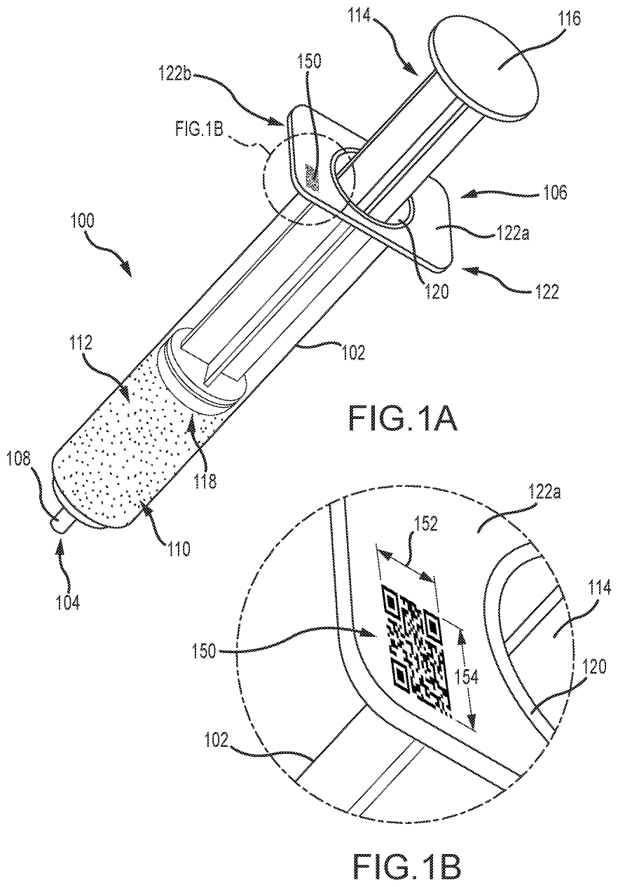

Turning now to the figures, FIGS. 1A and 1B depict a first embodiment 100 of a container according to the present invention. The container 100 generally includes a syringe body 102 extending longitudinally between and including a distal end 104 and a proximal end 106. Disposed at the distal end 104 may be a tip 108, which may include a hypodermic needle to be inserted into a human or other animal, an irrigation tip, or a coupling (e.g. Luer lock or fitting) to be coupled to another device (such as a syringe body 102 formed as a pre-filled syringe cartridge adapted to be coupled to a mating interface, such as a needle or irrigation tip (not shown)). The syringe body 102 further includes a barrel 110 disposed between the distal end 104 and the proximal end 106. The barrel 110 is adapted to contain a substance 112 to be forced or extracted through the tip 108. Situated at least partially within the barrel 110 may be a plunger 114 extending from a proximal end 116 disposed outside the barrel 110 to a distal end 118 disposed within the barrel 110. The plunger 114 may extend into the barrel 110 through an open proximal end 120 thereof. At least partially surrounding a portion of the barrel 110 may be a dispensing support structure, such as a flange 122 radially extending from the barrel 110 and longitudinally disposed closer to the proximal end 106 of the syringe body 102 than to the distal end 104 of the syringe body 102. The flange 122 preferably has opposing proximal 122a and distal 122b surfaces.

The syringe body 102 is preferably formed of a unitary, one-piece construction, such as by being injection molded. The body 102 (or any portion of a container on which a code 150 is placed) may be composed essentially of or include plastic (e.g., polyvinyl chloride (PVC), polypropylene, ethylene vinyl acetate (EVA or EVAM), polyester, copolyester, cyclic olefin polymer or copolymer (COP or COC), polyvinylidene fluoride (PVDF), polycarbonate, or any other plastic resin), metal (copper, aluminum, steel, nickel, gold, platinum), and/or glass.

Disposed preferably on an outer surface of the syringe body 102 or the plunger 114 is a code 150. Although ink could be used to apply the code 150 (e.g., the code 150 could be printed), the code 150 is preferably indelibly etched into the material of the container 100. Etching may be done with a laser, or a chemical mask process such as photoetching. In the case of an etched code, then, the underlying substrate (e.g., syringe body 102 or plunger 114) actually forms the code, having been altered by a laser or chemical, for example. The code 150 may be any code but is preferably a machine readable code (i.e., any technology that conveys information linked to a container or substance in a form that can be entered into an electronic record, such as a patient record, or other computer system via an automated process), such as a linear (1-dimensional) barcode (e.g., a universal product code (UPC)), or a matrix (2-dimensional) barcode (e.g., a quick response (QR) code), the latter being most preferred. Examples of preferred codes may be found in U.S. Pat. No. 8,651,385, issued to Prpa, which is incorporated by reference herein in its entirety. The code 150 preferably includes a code length 152 and a code width 154, each of which may be between about one millimeter and about 30 millimeters. Most preferably, the code length 152 and code width 154 are substantially equal to form a QR code of about one millimeter by about one millimeter to about 5 millimeters by about 5 millimeters. In addition to the machine readable code, human readable plain-text may be provided for the same or similar purposes. The code may be issued under a standardized system, such as a system operated by a regulatory agency (e.g., U.S. Food and Drug Administration), and may conform to international standards, such as those promulgated by the International Organization for Standardization (ISO), namely ISO/IEC 15459-2 (Unique identifiers: Registration procedures), -4 (Unique identifiers: Individual items), and -6 (Unique identifier for product groupings), and ISO/IEC 646 (ISO 7-bit coded character set for information exchange). An example of such code may be a code assigned, issued, and/or defined by a governing or oversight body (e.g., a unique device identifier (UDI) as defined by the U.S. Food and Drug Administration) or similar code meeting substantially similar or identical formalities.

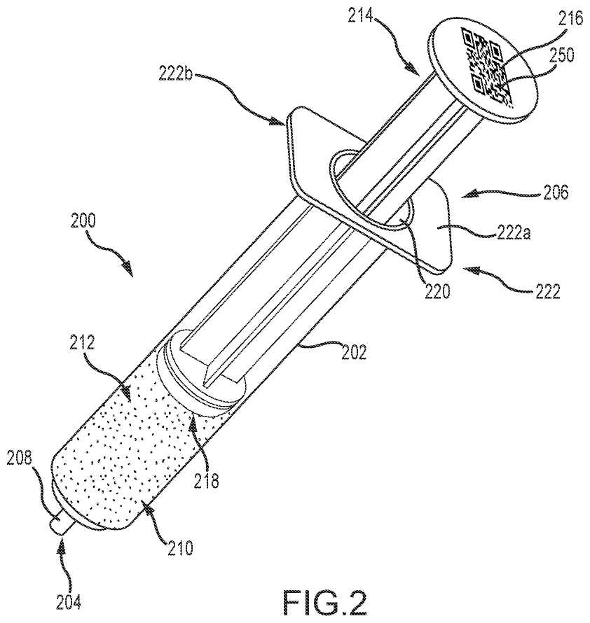

FIG. 2 is a perspective view of a second embodiment 200 of a container according to the present invention, where like numerals represent at least substantially similar or identical structure to the first embodiment 100. In this embodiment 200, the code 250 is placed on or etched into the plunger 214, such as by being etched into the proximal end 216 thereof.

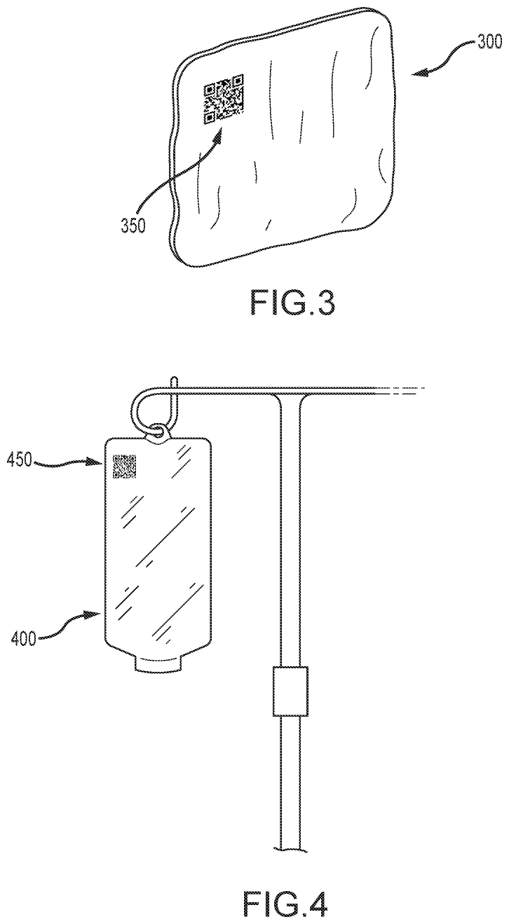

FIG. 3 depicts a representative embodiment 300 of a container or carrier, such as a transdermal patch. Within the patch 300 may be a substance intended to be delivered transcutaneously, such as testosterone, nicotine, cannabinoids, analgesic, contraceptive, nitroglycerin, or local anesthetic. The patch 300 includes a code 350 according to the present invention.

FIG. 4 shows another embodiment 400 of a container, a gravity feeding bag, according to the present invention. This type of container 400 may contain a fluid to be delivered intravenously. The fluid may be a simple saline solution or a complex nutrition cocktail, for example. Other fluid may include blood, opiates, opioids, non-steroidal anti-inflammatory drugs (NSAIDs) (e.g., acetaminophen, ibuprofen, naproxen sodium), antidepressants, diuretics, insulin, anticoagulants, steroids, adrenaline, antihistamines, coagulation modifiers, radiologic agents, etc. The container 400 includes a code 450 according to the present invention.

Not only can embodiments of the present invention be used in conjunction with containers that remain ex vivo before, during and after administration of a substance contained therein, FIG. 5 depicts an embodiment 500 of a container to be consumed in vivo, such as by being swallowed. This container 500 is a capsule 502, such as a gelatin or plant-derived capsule, which may contain a medicine, vitamin, or food supplement, for example. The capsule 502 may have a first half 504 and a second half 506 of conventional manufacture, generally assembled along a longitudinal axis 508. Not of conventional capsule manufacture, however, is a code 550 provided on one or each half. The capsule 502 may be of any desirable size, such as size 000 (about 26 millimeters long) to size 5 (about 11 millimeters long). In any event, the code has a length 552 along the longitudinal axis 508 that is about 5% to about 20% of the entire capsule length.

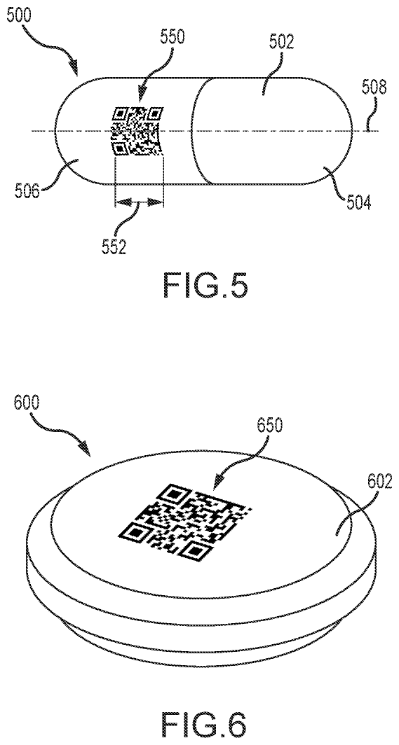

Further, instead of a container being marked, an in vivo consumable composition itself may be coded, such as that shown in FIG. 6. FIG. 6 depicts an embodiment 600 of a consumable, digestable substance in the form of a tablet 602, which may be formed by compression of a granulated material, such as a pharmaceutical material intended to be absorbed by an animal digestive system, by a conventional tablet press. The tablet 602 preferably has a substantially or completely homogenous cross section of material. Alternatively, a pressed tablet may be sealed and/or coated and/or polished. After or during pressing, the code 650 may be applied to or etched into the tablet 602. A further material that may be coded according to the present invention is ingestible film, such as that disclosed in U.S. Pat. No. 7,357,891, issued to Yang et al., and incorporated herein by reference in its entirety.

A code (e.g. 150) according to the present invention may include encoded information about a substance to be taken in vivo. The code is preferably an indelible marking on the container or the substance, and preferably applied without ink, such as by laser etching. The code may be encoded with information related to the substance itself, such as a brand name, generic name, National Drug Code (NDC) number, ingredients, amount/quantity of substance (e.g., volume or mass), possible side effects, indications, contraindications, and/or appearance (e.g., round white tablet, chalky pink liquid, etc.), or any combination thereof. Encoded information may also or alternatively relate to manufacturing and/or storage of the substance, including a manufacturer's lot number, production date, production time of day, and/or expiration date, or any combination thereof. If the code is placed on a container, the code may include encoded information related to the container itself, including capacity, type (e.g., syringe, IV bag, transdermal patch, single serve sealed cup or pouch), manufacturing and/or storage of the container, including manufacturer's lot number, container serial number, production date, production time of day, expiration date, and/or compatible contents, or any combination thereof. The code may additionally or alternatively include encoded information related to a particular natural or juristic person, such as a prescribing physician, pharmacist, administering physician, patient, insurance company, and/or consumer, or any combination thereof. Information related to a particular person may be name, postal address, telephone number, e-mail address, health insurance company name, health insurance policy number, social security number or other government issued identifier, credit card information or other payment account information, birth date, and/or age, prescribed or allowed dosage amount, prescribed or allowed dosage schedule, or any combination thereof.

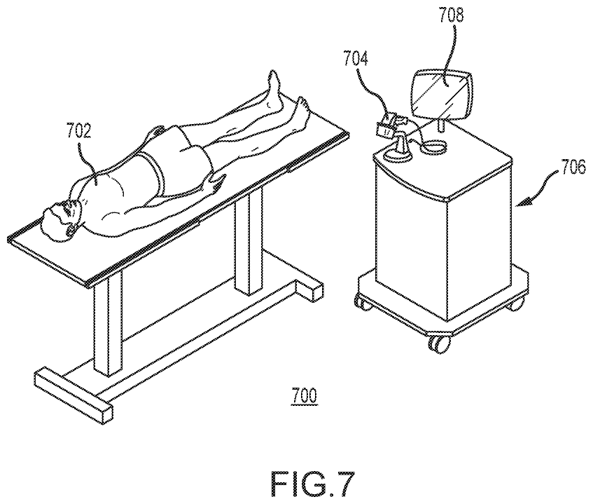

FIG. 7 is a perspective view of an administration environment 700 in which embodiments of devices according to the present invention may be used. Such environment may be a hospital patient room, operating room, cafeteria, restroom, pharmacy, doctor's office, etc. The embodiment shown includes an environment in which medication may be administered to a patient 702. The environment may or may not include a sterile field. The patient 702 may be conscious or unconscious, but generally is to be provided with some substance to be taken in vivo, whether orally, intravenously, transcutaneously, or percutaneously. In the environment 700 is preferably a scanner 704, which may be used in accordance with the present invention. The scanner 704 may be any scanner adapted and configured to read a code (e.g. 150) and deliver an electronic representation of the code to a computer 706, which may be connected to a network, such as a local area network (LAN), wireless local area network (WLAN), wide area network (WAN), and/or a global computer network, such as the Internet. The scanner 704 may be a laser barcode reader or an image-based barcode reader, for example, which may be handheld or securely disposed within a housing. The computer 706 preferably includes or is communicatively coupled to a display 708 and may further include a speaker or buzzer (not shown) for providing feedback to a user.

A method of substance (e.g., food and/or drug) administration control or verification according to the present invention includes, in an administration environment (e.g., 700) including an animal (e.g., human, feline, canine, livestock) to receive the substance, receiving a container (e.g., 100) including a code (e.g., 150) disposed thereon and containing the substance (e.g., 112), scanning the code with a scanner (e.g., 704), and delivering the substance to the animal. The delivery of the substance may be direct insertion of the substance into the body of the animal (e.g., intravenous injection), or handing the container (e.g., a capsule 500) or the substance (e.g., a tablet 600) to the animal for oral consumption. The method may further include the step of, after scanning the code with the scanner, receiving feedback either directly from the scanner or from an electronic device (e.g., a computer 706) in communication with the scanner. The feedback may be negative feedback (indicating that the delivery step should not occur at this time) or positive feedback (indicating that the delivery step should be performed at this time). The method may then proceed with the delivery step, preferably only if the feedback was positive feedback, thus including the step of withholding the substance from the animal if the feedback is negative feedback. The method may further include a step of scanning the container after the delivery step, which may indicate that the delivery is complete. Prior to or after (but substantially contemporaneous with) each scanning step, a substance administrator (e.g., nurse, doctor, animal itself) may be required to identify itself by entering a password or personal identification number (PIN) into the computer.

A data record is preferably created as a result of each scanning step. The data record preferably includes an association of the information encoded by the code along with a date and a time of day, reflecting the date and time of the scanning step. The data record may be static (i.e., automatically generated with a timestamp and/or location stamp), or a user may be prompted for data entry related to the scan. For instance, a user may be prompted to enter a purpose for the scan or a purpose may be automatically entered based on the location of the scanner. Scan purposes may include LOCATION LOG, IDENTIFICATION, RX FILL, ADMINISTRATION, ADMINISTRATION START, ADMINISTRATION END, or DISPOSAL, for example. A LOCATION LOG purpose may be selected (manually or automatically) if the purpose of the scan is simply to log the location (e.g., in a database) of the scanned container or substance, such as a geographic location tracked by GPS coordinates, a location indicated by postal address, floor number, room number, or even a location along a manufacturing line. An IDENTIFICATION purpose may be selected (manually or automatically) if the purpose of the scan is to identify the container and/or substance, in which case information related to the container and/or substance may be supplied to a user after a query is made to a database. An RX FILL purpose may be selected (manually or automatically) if the purpose of the scan is to log a location and/or timestamp of setting the container and/or substance aside for a particular patient, or for dispensing the container and/or substance from a pharmacy or other storage location. An ADMINISTRATION purpose may be selected (manually or automatically) if the purpose of the scan is to verify the propriety of administering or providing the substance to a patient and/or for logging at least an approximate location and/or timestamp of such administration. ADMINISTRATION START and END purposes may be selected (manually or automatically) when a container is scanned at or near the time a substance is started and stopped, respectively, from being administered to a patient. Such purposes may include an indication of the propriety of the administration and/or for logging an at least approximate location and/or at least approximate timestamps of starting and stopping such administration. While ADMINISTRATION START and END purposes may be used at a single point of administration (e.g., when a substance is delivered and placed in vivo within a short time (a few seconds to a few minutes) after the scan), they may prove more useful when the substance is administered from the container over a longer period of time, such as through a drip IV. A DISPOSAL purpose may be selected (manually or automatically) if the purpose of the scan is to indicate that the container and/or substance is being disposed of, such as by being placed in a sharps container, in the trash, or in an incinerator, for example, and is not to be taken in vivo. The purposes may not be mutually exclusive. That is, for an ADMINISTRATION purpose, a query may first be made to a database to identify what the container and/or substance was previously

The code (e.g. 150) may be used in an environment other than the administration environment 700, so as to track a particular container or substance. For instance, the code may be scanned, and a data record created, in a pharmacy, food kitchen, manufacturing plant (possibly at multiple inspection locations), dispensary, etc.

There are several uses for coded containers and/or substances. One method, as described above, is for substance administration control, verification and/or confirmation. Another method according to the present invention may be an invoicing method. Such invoicing method may include steps of accessing a data record resulting from a scanning step (as described above) and generating an invoice or an invoice line item including a description of the substance and a respective price, to streamline a billing process. Inventory management may be another method using containers and/or substances marked according to the present invention.

Another method according to the present invention includes verification by and/or control of a dispensing machine in a manufacturing environment in which containers (i.e., syringes, syringe cartridges, capsules, IV bags, etc.) are filled. For example, syringes intended to receive a predetermined dose of a predetermined substance may be marked with a code as described herein. Such marked syringes may be presented to a manufacturing line individually or in bulk, such as through a hopper, and oriented to receive the predetermined substance. Prior to the syringe barrel receiving the substance, the code on the syringe may be scanned and the encoded information used to determine which substance and the amount of that substance to place in the syringe. This information may be used in selecting a substance from a plurality of potential substances loaded into such dispensing machine, where the selected substance matches a substance identified by the code scan.

Additionally, a container of the substance loaded into the dispensing machine may also include a code. The syringe may then be filled if the encoded information indicates that the type of substance loaded into the dispensing machine is appropriate, or prevented from being filled if the substance is not appropriate. Thus, a fill/no-fill determination may be automatically made by a dispensing machine (which includes a computer including or communicating with a scanner (e.g. 704)) based on a comparison of information encoded in a scanned code on a syringe to information related to the substance loaded into, and ready for dispensing from, a dispensing machine. This comparison may be made between information obtained or related to the scanned code and information independently maintained or accessible to the dispensing machine. The information independently maintained by or accessible to the dispensing machine may be manually entered into a database or data field accessible by the dispensing machine, or may be automatically accessed after a scan is made of a bulk container code. While it is preferred that at least a portion of the information associated with each of the syringe code and the bulk container code match (e.g., substance identification), it is not necessary for all encoded information related to the two codes match to proceed with filling.

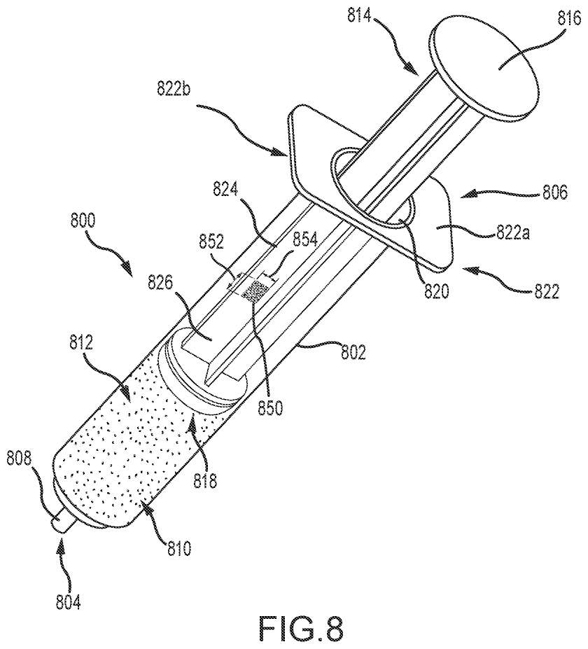

FIG. 8 illustrates a fourth embodiment 800 of a container according to the present invention. Similar to the first embodiment container 100, the fourth embodiment 800 preferably includes a syringe body 802 extending longitudinally between and including a distal end 804 and a proximal end 806. Disposed at the distal end 804 may be a tip 808, which may include a hypodermic needle to be inserted into a human or other animal, an irrigation tip, or a coupling (e.g. Luer lock or fitting) to be coupled to another device (such as a syringe body 802 formed as a pre-filled syringe cartridge adapted to be coupled to a mating interface, such as a needle or irrigation tip (not shown)).

The syringe body 802 preferably further includes a barrel 810 disposed between the distal end 804 and the proximal end 806. The barrel 810 is adapted to contain a substance 812 to be forced or extracted through the tip 808. Situated at least partially within the barrel 810 may be a plunger 814 having a shaft 824 extending from a proximal end 816 disposed outside the barrel 810 to a distal end 818 disposed within the barrel 810. The plunger 814 may extend into the barrel 810 through an open proximal end 820 thereof. At least partially surrounding a portion of the barrel 810 may be a dispensing support structure, such as a flange 822 radially extending from the barrel 810 and longitudinally disposed closer to the proximal end 806 of the syringe body 802 than to the distal end 804 of the syringe body 802. The flange 822 preferably has opposing proximal 822a and distal 822b surfaces.

The syringe body 802 is preferably formed of a unitary, one-piece construction, such as by being injection molded. The body 802 is preferably transparent and may be composed essentially of or include transparent plastic and/or glass as is known in the art.

Disposed preferably on a surface 826 of the shaft 824 of the plunger 814 is a code 850. Although ink could be used to apply the code 850 (e.g., the code 850 could be printed), the code 850 is preferably indelibly etched into the material of the shaft 824. Etching may be done with a laser, or a chemical mask process such as photoetching. In the case of an etched code, then, the underlying substrate (e.g., the shaft 824 of the plunger 814) actually forms the code, having been altered by a laser or chemical, for example.

The code 850 may be any code but is preferably a machine-readable code (i.e., any technology that conveys information linked to a container or substance in a form that can be entered into an electronic record, such as a patient record, or other computer system via an automated process), such as a linear (1-dimensional) barcode (e.g., a universal product code (UPC)), or a matrix (2-dimensional) barcode (e.g., a quick response (QR) code), the latter being most preferred. Examples of preferred codes may be found in U.S. Pat. No. 8,651,385, issued to Prpa, which is incorporated by reference herein in its entirety.

The code 850 preferably includes a code length 852 and a code width 854, each of which may be between about one millimeter and about 30 millimeters. Most preferably, the code length 852 and code width 854 are substantially equal to form a QR code of about one millimeter by about one millimeter to about 5 millimeters by about 5 millimeters. In addition to the machine-readable code, human readable plain-text may be provided for the same or similar purposes. The code may be issued under a standardized system, such as a system operated by a regulatory agency (e.g., U.S. Food and Drug Administration), and may conform to international standards, such as those promulgated by the International Organization for Standardization (ISO), namely ISO/IEC 15459-2 (Unique identifiers: Registration procedures), -4 (Unique identifiers: Individual items), and -6 (Unique identifier for product groupings), and ISO/IEC 646 (ISO 7-bit coded character set for information exchange). An example of such code may be a code assigned, issued, and/or defined by a governing or oversight body (e.g., a unique device identifier (UDI) as defined by the U.S. Food and Drug Administration) or similar code meeting substantially similar or identical formalities.

The foregoing is illustrative only of the principles of embodiments according to the present invention. Modifications and changes will readily occur to those skilled in the art, so it is not desired to limit the invention to the exact disclosure herein provided. While the preferred embodiment has been described, the details may be changed without departing from the invention, which is defined by the claims.

We claim:

1. A system comprising:
a container for containing a consumable substance and configured to be consumed by an animal to deliver the consumable substance to the animal;
wherein the container itself is digestible by the animal;
a machine-readable code indelibly placed on the container by laser etching; and
wherein the machine-readable code is encoded with data representative of information related to at least one of the container, the consumable substance contained within the container, or the animal to which the substance is to be administered.

2. A system according to claim 1, wherein the information related to the consumable substance contained within the container includes at least one of substance ingredients, possible side effects caused by the substance, a substance production date, a substance production time of day, or a substance expiration date.

3. A system according to claim 1, wherein the information related to the container includes at least one of a container capacity, a container type, a manufacturer lot number for the container, a container serial number, a container production date, a container production time of day, a container expiration date, or a list of compatible contents.

4. A system according to claim 1, wherein the information related to the animal to which the substance is to be administered includes at least one of a postal address, a telephone number, an e-mail address, a health insurance company name, a health insurance policy number, a social security number, a payment account, a birthdate, age, prescribed dosage, or prescribed dosage schedule.

5. A system according to claim 1, wherein the machine-readable code is a one-dimensional machine-readable code.

6. A system according to claim 1, wherein the machine-readable code is a two-dimensional machine-readable code.

7. A system according to claim 6, wherein the machine-readable code is a quick response (QR) code.

8. A system according to claim 1, wherein the machine-readable code is further encoded with data representative of information related to a name of at least one of a prescribing physician, a pharmacist filling a prescription of the consumable substance, or an administering physician.

9. A system according to claim 1, wherein the container comprises a capsule.

10. A system according to claim 9, wherein the capsule comprises a first half and a second half.

11. A system according to claim 11, wherein the machine-readable code is provided on one of the first half or the second half.

12. A system according to claim 11, wherein the machine-readable code is provided on both the first half and the second half.

13. A system comprising:
a consumable composition of a substance configured to be consumed by an animal;
a machine-readable code indelibly placed on the consumable composition by laser etching; and
wherein the machine-readable code is encoded with data representative of information related to at least one of the consumable composition or the animal by which the consumable composition is to be consumed.

14. A system according to claim 13, wherein the information related to the consumable composition includes at least one of composition ingredients, possible side effects caused by the composition, a composition production date, a composition production time of day, or a composition expiration date.

15. A system according to claim 13, wherein the information related to the animal to which the composition is to be administered includes at least one of a postal address, a telephone number, an e-mail address, a health insurance company name, a health insurance policy number, a social security number, a payment account, a birthdate, age, prescribed dosage, or prescribed dosage schedule.

16. A system according to claim 13, wherein the machine-readable code is a one-dimensional machine-readable code.

17. A system according to claim 13, wherein the machine-readable code is a two-dimensional machine-readable code.

18. A system according to claim 17, wherein the machine-readable code is a quick response (QR) code.

19. A system according to claim 13, wherein the machine-readable code is further encoded with data representative of information related to a name of at least one of a prescribing physician, a pharmacist filling a prescription of the consumable substance, or an administering physician.

20. A system according to claim 13, wherein the consumable composition is in the form of a tablet.
